(19) United States
(12) Patent Application Publication
(10) Pub. No.: US 2012/0310060 A1
(43) Pub. Date: Dec. 6, 2012

(54) METHOD OF ANALYZING PHOTON DENSITY WAVES IN A MEDICAL MONITOR

(75) Inventors: Clark R. Baker, JR., Newman, CA (US); Youzhl Li, Longmont, CO (US); Andy S. Lin, Boulder, CO (US); Daniel Lisogurski, Boulder, CO (US)

(73) Assignee: Nellcor Puritan Bennett LLC, Boulder, CO (US)

(21) Appl. No.: 13/149,578

(22) Filed: May 31, 2011

(51) Int. Cl. A61B 5/1455

(52) U.S. Cl. 600/324

(57) ABSTRACT

A monitoring system may include an emission feature capable of emitting light into tissue, a modulator capable of modulating the emitter at a modulation frequency, e.g., in a range of about 10 MHz to 3.0 GHz, to generate resolvable photon density waves, a detection feature capable of detecting photons of the photon density waves after passage through the tissue, and a processor capable of using phase and amplitude differences of the photon density wave signal relative to a reference to determine one or more physiological parameters. The phase and amplitude differences may be much lower frequency that the modulation rate. Accordingly, these differences may be masked by signal artifacts. Provided herein are signal conditioning techniques that may improve the signal to noise ratio of photon density wave signals and yield a more robust phase and amplitude signal.

### BACKGROUND

The present disclosure relates generally to a tissue analysis system that utilizes emission and detection of photon density waves to assess tissue characteristics, and, more particularly, to a system for evaluating scattering properties of tissue based on distribution of photons in photon density waves emitted into the tissue and features of the photon density waves detected after passing through the tissue.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Photoplethysmography is a non-invasive monitoring technique that involves emitting light at one or more specific wavelengths into a patient's tissue and detecting the light after it has passed through the tissue. Photoplethysmography may be used to monitor a patient's respiration rate, respiration effort, CNIBP, and many other measurements that are related to blood flow. One example of a photoplethysmography-based monitoring technique is pulse oximetry. For example, pulse oximetry may be used to measure blood oxygen saturation of hemoglobin in a patient's arterial blood and/or the patient's heart rate. Specifically, these blood flow characteristic measurements may be acquired using a non-invasive sensor that passes light through a portion of a patient's tissue and photo-electrically senses the light through the tissue. Typical pulse oximetry technology currently utilizes two light emitting diodes (LEDs) that emit different wavelengths of light and a single optical detector to measure the pulse rate through and oxygen saturation of a given tissue bed.

A typical signal resulting from the sensed light may be referred to as a plethysmographic waveform. It should be noted that the amount of arterial blood in the tissue is generally time varying during a cardiac cycle, which is reflected in the shape of plethysmographic waveforms. Such measurements are largely based on absorption of emitted light by specific types of blood constituents and do not specifically take scattering into account. Indeed, traditional pulse oximeters make measurements based on a manipulation of the Lambert-Beer Law, and commonly assume that the two different wavelengths of light from light emitters travel the same path length through the same tissue. Thus, scattering differences are essentially not taken into account. However, once acquired, absorption measurements, as typically acquired by traditional pulse oximeters, may be used with various algorithms to estimate a relative amount of blood constituent in the tissue. For example, such measurements may provide a ratio of oxygenated to deoxygenated hemoglobin in the volume being monitored.

The accuracy of blood flow characteristic estimation via pulse oximetry depends on a number of factors. For example, variations in light absorption characteristics can affect accuracy depending on where the sensor is located and/or the physiology of the patient being monitored. Additionally, various types of noise and interference can create inaccuracies. For example, electrical noise, physiological noise, and other interference can contribute to inaccurate blood flow characteristic estimates. Some sources of noise are consistent, predictable, and/or minimal, while some sources of noise are erratic and cause major interruptions in the accuracy of blood flow characteristic measurements. Accordingly, it is desirable to enable more accurate and/or controlled measurement of physiologic parameters by providing a system and method that takes path length and tissue scattering properties into account, and that addresses inconsistencies in physiologic characteristics of patients and issues relating to noise.

### BRIEF DESCRIPTION OF THE DRAWINGS

Advantages of the disclosed techniques may become apparent upon reading the following detailed description and upon reference to the drawings in which:

FIG. 1 illustrates a perspective view of a photoplethysmography system capable of utilizing photon density waves in accordance with present embodiments;

FIG. 2 illustrates a block diagram of a pulse oximeter system capable of utilizing photon density waves in accordance with present embodiments;

FIG. 3 illustrates an example of a source modulation signal in accordance with present embodiments;

FIG. 4 is a graph showing a mean path length and amplitude measurement from a photon density wave sensor; and

FIG. 5 is a flow diagram of a method for determining a physiological parameter.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Present embodiments relate to systems and sensors for acquiring signals from photon density wave devices and correlating these signals to physiological parameters. In particular embodiments, these sensors and systems may be configured to perform photoplethysmography. The techniques provided herein may assess variations in photon path lengths and/or scattering properties, either between persons, tissues, wavelengths, or over time. Such information may be used in measurements of blood or tissue constituent concentrations and may be used in conjunction with monitoring of a patient's respiration rate, respiration effort, CNIBP, and blood flow measurements. In one particular embodiment, the systems provided herein may be used to perform pulse oximetry. Traditional pulse oximetry sensors and photon density wave-configured sensors emit light into tissue and detect the light that has passed through the tissue. Both types of sensors are capable of detecting changes in the amplitude of the emitted light and relating these changes to physiological information. However, the photon density wave-configured sensors as provided herein are capable of acquiring signals that not only provide amplitude information but that also provide phase shift or phase delay information. The addition of phase information to the detected signal allows a calculation of the path length of the detected light.

This calculated path length represents an improvement over systems that estimate a path length or that, in multi-wavelength systems, assume that the path length of the individual wavelengths (e.g., red and IR) are the same in calculations of physiological parameters from optical signal, which in turn may lead to greater accuracy in determining physiological parameters. For example, in pulse oximetry applications, it may be assumed that the path lengths of the red and IR light are the same. But this assumption breaks at low saturation level, causing deteriorated accuracy. As provided herein, a direct calculation of path length may result in improved measurement accuracy. In addition, the path length calculations may be applied to compensate for path length differences among the different wavelengths in measuring regional saturation in order to improve accuracy.

Provided herein are systems and sensors configured to modulate the emitted light at sufficiently high frequencies to generate resolvable photon density waves that are passed through tissue and detected for analysis of changes in the characteristics of an emitted and detected photon density wave. Generation of the resolvable photon density waves may include essentially turning light sources on and off at a high frequency (e.g., 10-3000 megahertz range). In particular, the photon density wave-configured sensors as provided herein acquire relatively high frequency signals because the emitted light is modulated at high frequency. However, the phase and amplitude differences of the detected signals relative to the emitted light are much lower frequency than the modulation rate. In one embodiment, the light source modulation occurs at very high frequency but the amplitude and phase differences in the received signal only change at rates related to the volume of blood or other materials through which the light passes. For example, typical changes in amplitude are band-limited to less than 40 Hz. Accordingly, processing the acquired signals from detected photon density waves may present certain challenges because these relatively small phase and amplitude differences may be masked by the signal artifacts. Provided herein are techniques for processing the acquired signals that minimize artifacts and improve signal quality. In certain embodiments, these signal processing techniques may be applied to the phase-delay signal as well as the amplitude signal to reduce motion artifacts, electronic interference, and/or artifacts of physiological origin. The present disclosure is directed to evaluating a time-delay (i.e., phase-delay) of photon density waves, which may be enhanced using particular signal processing techniques.

In particular, the acquired phase-delay signals exhibit indications of arterial pulse as well as non-pulsatile components. With regard to the phase signals, application of certain types of signal processing may effect a more robust determination of tissue optical path length. Further, while the signal conditioning may occur in the digital domain, additional processing steps may also be employed in the analog domain. In one embodiment, single or multiple channel sigma delta converters may be use to digitize the phase and amplitude signals. After additional processing steps in the digital domain, the detected phase and amplitude signals may be compared to the reference signal to determine phase differences of the detected light that are related to the optical path length and/or a physiological parameter (e.g., a clinical condition). Alternatively, the detected phase and amplitude signals may be analyzed over time to determine relative changes. In such an embodiment, the detected phase and amplitude signals may not necessarily be compared to a reference signal.

In an alternative embodiment, the phase difference and amplitude difference signals may be resolved in the analog domain by comparing the photon density wave signal to a reference prior to digitization. The signal processing techniques as provided herein may be applied directly to the photon density wave signal or to the phase difference over time (e.g. the phase or phase-delay signal) or amplitude difference over time (e.g., the amplitude or amplitude difference signal).

A change in phase of a photon density wave signal may correspond to scattering components of the tissue (in addition to minor factors such as the speed of light in blood vs., tissue), while a change in amplitude may correspond to absorptive components in the tissue. These relationships may be used to determine one or more physiological parameters. For example, since the scattering coefficient may change over time depending on a total quantity of erythrocytes in the tissue, variations in phase changes may correspond to variations in total hemoglobin. Thus, such changes in phase over time may be due predominantly to the total number of scattering particles (e.g., total hemoglobin), and not merely a ratio of particles (e.g., oxygenated and total hemoglobin). Changes in amplitude of the photon density wave signals may correspond to the absorptive components of the pulsatile patient tissue, not scattering components. Certain components of the tissue may absorb different wavelengths of light, such as red or infrared light, in different amounts. By analyzing decreases in amplitudes of the received single-wavelength photon density wave signals, a ratio of different types of particles in the pulsatile patient tissue, such as oxygenated and deoxygenated hemoglobin, may be estimated. With measurements of scattering and absorption characteristics of the tissue, physiological parameters such as regional oxygen saturation, total hemoglobin, perfusion, and other vascular conditions may be obtained.

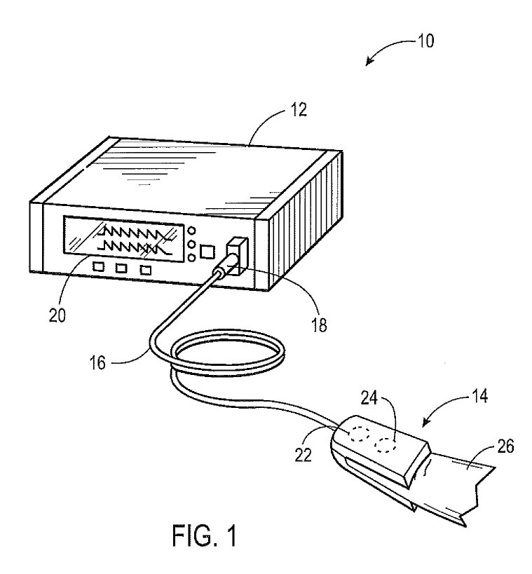

FIG. 1 illustrates a perspective view of a photon density wave pulse oximetry system 10, which may include a patient monitor 12 that communicatively couples to a pulse oximetry sensor 14. Although the depicted embodiment relates to pulse oximetry, it should be understood that systems, monitors, and sensors as provided herein may be directed to other types of medical monitoring. In particular, such systems may use at least one light source. A sensor cable 16 may connect the patient monitor 12 to the sensor 14, and may include two fiber optic cables. One of the fiber optic cables within the sensor cable 16 may transmit a multi-wavelength (or one wavelength) photon density wave input signal from the patient monitor 12 to the sensor 14, and another of the fiber optic cables may transmit a multi-wavelength photon density wave output signal from the sensor 14 to the patient monitor 12. The cable 16 may couple to the monitor 12 via an optical connection 18. Based on signals received from the sensor 14, the patient monitor 12 may determine certain physiological parameters that may appear on a display 20. Such parameters may include, for example, a plethysmogram or numerical representations of patient blood flow (e.g., partial oxygen saturation or a measurement of total hemoglobin). The monitor 12 may also include a tangible computer-readable medium (e.g., a memory, a floppy disk, or a CD), a processor, and various monitoring and control features).

The patient monitor 12 may modulate light sources of one or more wavelengths at modulation frequencies of approximately 10 MHz-3 GHz, which may produce resolvable photon density wave signals in pulsatile tissue because the resulting photon density waves at such frequencies may have wavelengths shorter than a mean absorption distance in pulsatile tissue. In some embodiments, the patient monitor 12 may sweep the modulation frequency of one or more of the light sources in a range from 50 MHz to 2.4 GHz. Some embodiments of the patient monitor 12 may be configured to modulate between 100 MHz and 1 GHz or to sweep a range from 100 MHz to 1 GHz. The patient monitor 12 may, in certain embodiments, modulate the light sources primarily at a frequency of approximately 500 MHz. Other ranges are also possible.

In embodiments in which two light sources are used, the patient monitor 12 may multiplex these multiple single-wavelength photon density wave signals into a single multi-wavelength photon density wave signal, which may be provided to the sensor 14 via the sensor cable 16. Alternatively, several fibers may be used, each connected to a laser of different wavelength or more than one fiber could be used to provide two different distances between the emitter and detector at the sensor. The sensor 14 may include an emitter output 22 and a detector input 24. The emitter output 22 may guide the multi-wavelength photon density wave signal from the sensor cable 16 to enter pulsatile tissue of a patient 26. The detector input 24 may receive the resulting multi-wavelength photon density signal from the pulsatile tissue of the patient 26 and guide the received signal back to the patient monitor 12 via the sensor cable 16. The sensor 14 may be, for example, a reflectance-type sensor or a transmission-type sensor. Further, the sensor 14 may be applied to a patient's finger, ear, forehead, toe, or other suitable measurement site.

When the resulting photon density wave signal reaches the patient monitor 12, wave characteristics of the received photon density signals may be measured in accordance with present embodiments, and may include characteristics that relate predominantly to absorption of the emitted light in the probed medium (e.g., amplitude change) and characteristics that relate predominantly to scattering in the probed medium (e.g., phase shift). The correlation of certain wave characteristic (e.g., amplitude and phase) measurements to certain medium characteristics (e.g., quantity of scattering particles and blood oxygen saturation) may depend on the modulation of the light sources within the patient monitor, which may generate resolvable photon density waves. Specifically, to produce resolvable photon density waves, the modulation frequency of such signals should produce photon density waves having modulation wavelengths that are shorter than a mean absorption distance of the probed tissue medium.

As indicated above, the system 10 may be utilized to make measurements that relate predominantly to scattering in the observed volume. More specifically, the system 10 may be utilized to make measurements relating to a total amount of scattering particles in the observed volume based on phase shifts detected in the emitted light waves. For example, the system 10 may emit light that is modulated at a frequency (e.g., 10 MHz to 3 GHz) sufficient to generate resolvable photon density waves, and then measure the phase shift of these waves to facilitate estimation of a total number of scattering particles in the observed medium. Similarly, as set forth above, the system 10 may be utilized to make measurements that relate predominantly to absorption in an observed volume or to facilitate detection of a ratio of certain constituents in the blood (e.g., a ratio of oxygenated hemoglobin to the total hemoglobin). It should be noted that the amplitude changes and phase shifts measured at a detection point may be considered relative to one or more points. For example, the amplitude and phase shifts measured from the detector input may be considered relative to the associated values generated at the emitter output.

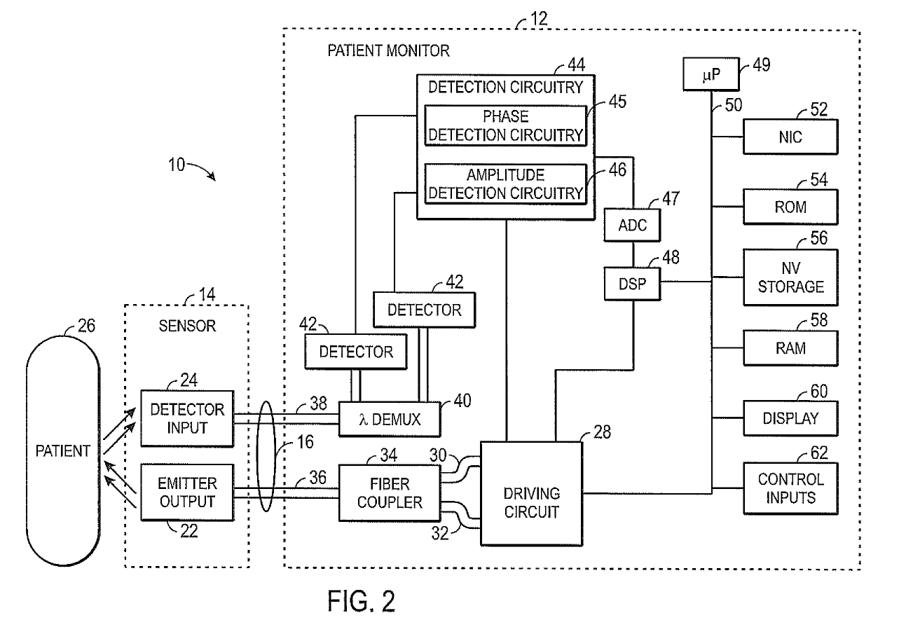

FIG. 2 represents a block diagram of the system 10 of FIG. 1. As illustrated in FIG. 2, the patient monitor 12 may generate a photon density wave signal using a driving circuit 28, which may include one or more light sources. In the depicted embodiment, the system 10 is shown as having at least two light sources. However, it should be understood that the system 10 may be configured to operate with only one light source. Further, at least one wavelength may be used for hemoglobin measurement. For other parameters (SpO₂, rSO₂, at least two wavelengths of light may be used. Such wavelengths may include red wavelengths of between approximately 600-700 nm and/or infrared wavelengths of between approximately 800-1000 nm. By way of example, the light sources of the driving circuit 28 may be laser diodes that emit red or infrared light with wavelengths of approximately 660 nm or 808 nm, respectively. In some embodiments, the one or more light sources of the driving circuit 28 may emit three or more different wavelengths light. Such wavelengths may include a red wavelength of between approximately 620-700 nm (e.g., 660 nm), a far red wavelength of between approximately 690-770 nm (e.g., 730 nm), and an infrared wavelength of between approximately 860-940 nm (e.g., 900 nm). Other wavelengths that may be emitted by the one or more light sources of the driving circuit 28 may include, for example, wavelengths of between approximately 500-600 nm and/or 1000-1100 nm. In particular embodiments, the light source may include one or more LEDs that are configured to be controlled by the drive circuit 28 to generate photon density waves.

The driving circuit 28 may modulate these light sources at a modulation frequency between approximately 10 MHz to 3 GHz. Such modulation frequencies may suffice to produce resolvable photon density waves when emitted into pulsatile tissue of the patient 26, since corresponding wavelengths of the photon density waves may be shorter than a mean distance of absorption in the tissue. The modulation frequency of each light source may vary, as one light source may have a higher or lower modulation frequency than another light source. The driving circuit 28 may represent one or more components of commonly available drive circuits (e.g., DVD R/W driver circuits) for high-frequency modulation. Examples of such devices may include the LMH6525 available from National Semiconductor Inc. In embodiments in which CD/DVD laser driver components are employed, additional circuitry may be incorporated, such as filters, delay elements, or other calibration components, to achieve the predetermined amplitude and phase delay relationship between the drive signal and reference signal.

Other embodiments for driving the light source may be those disclosed in H-RM-02091 “Photon Density Wave Based Determination of Physiological Parameters” to Daniel Lisogurski et al., or H-RM-02039 “Photon Density Wave Based Determination of Physiological Parameters” to Youzhi Li, et al., both filed on May 31, 2011, the disclosures of which are incorporated by reference in their entirety herein for all purposes. For example, the drive circuit 28 may generate modulation signals and reference signals for use by other elements of the system 10 and receive instructions from a processor (e.g., processor 48 or 49) for generating these signals. In one embodiment, the drive circuit 28 may include a two-channel direct digital synthesis (DDS) component, such as Analog Devices AD9958. The drive circuit 28 may digitally synthesize a drive signal and reference signal at a matched predetermined frequency and waveform, such as a 400 MHz sine wave, although other waveforms and frequencies could be employed. The drive signal and reference signal may be synthesized with predetermined amplitude and phase delay relationships to each other. The amplitude of drive signal is synthesized based on the input parameters for the light source, possibly after further amplification, switching, or multiplexing by an RF switch. The amplitude of reference signal is synthesized based on the input parameters of the detection circuitry 44. The relative phase between the drive signal and the reference signal is synthesized based on the phase delay experienced by the drive signal through the sensor 14, cable 16, and tissue 26, among other factors, such as operating parameters of detection circuitry 44. For example, the phase delay synthesized by the drive circuit 28 may be calibrated to achieve a predetermined relative phase delay at detection circuitry 44, based in part on the length of optical cables 30 and 32.

In certain embodiments, an RF switch may provide switching, multiplexing, or buffering circuitry for selectively providing the drive signal. The RF switch may include a single-pole double-throw (SPDT) style of switch operable at the high frequencies of the drive signal to alternately provide the drive signal to its appropriate pathways in a repeating, sequential manner. The RF switch may also include a solid-state switch, such as transistors, RF junctions, diodes, or other solid state devices. In some examples, the RF switch receives switching instructions from the processor, while in other examples, a predetermined switching profile is included in the RF switch. In further examples, the RF switch includes signal conditioning components, such as passive signal conditioning devices, attenuators, filters, and directional couplers, active signal conditioning devices, amplifiers, or frequency converters, including combinations thereof. In yet further examples, the RF switch provides the drive signal to the appropriate pathways in a simultaneous manner. In any configuration of the RF switch, an “off” condition may be employed in which the drive signal is not provided.

In FIG. 2, the driving circuit 28 is illustrated to generate two single-wavelength photon density wave signals of different wavelengths respectively through an optical cable 30 and an optical cable 32. A fiber coupler 34 may join the two optical cables 30 and 32 together, multiplexing the two single-wavelength photon density wave signals into a multi-wavelength photon density wave signal. An optical cable 36, serving as an emitting cable, may carry the multi-wavelength photon density wave signal through the sensor cable 16 to the emitter output 22 of the sensor 14. The multi-wavelength photon density wave signal may thereafter enter pulsatile tissue of the patient 26, where the signal may be scattered and absorbed by various components of the tissue. The detector input 24 may receive and guide the portion of the signals reflected or transmitted through the patient tissue 26 to the patient monitor 12 over an optical cable 38 and any additional connectors, which may be a second of two optical cables of the sensor cable 16.

The received multi-wavelength photon density wave may be separated into its component light signals of various wavelengths by a wavelength demultiplexer 40. Using filters or gratings, for example, the wavelength demultiplexer 40 may split the received multi-wavelength photon density wave signal from optical cable 38 into received single-wavelength photon density wave signals that correspond to the emitted single-wavelength photon density wave signals originally produced by the driving circuit 28. In other words, the wavelength demultiplexer 40 may break the received multi-wavelength photon density wave signal into a first received signal at the first wave length (e.g., 660 nm) and a second received signal at the second wave length (e.g., 808 nm), which respectively may be analyzed by photodetectors 42. If the demultiplexer 40 is optical, then it may be present before the phase detection circuitry 44 as shown in FIG. 2. Alternatively, the signal may be demultiplexed via software methods, including Time Division Multiplexing (commonly used in pulse oximetry) as well as Frequency Division Multiplexing, transmitting the signals in Quadrature or using CDMA/Spread Spectrum techniques. These methods may eliminate the need for the optical demodulator and allow the software to demodulate the signals. Further, in single wavelength embodiments, the system 10 may not include a demodulation step, either in hardware or software.

The detectors 42 may receive, amplify, and convert these received single-wavelength photon density wave signals into corresponding electrical signals. Amplifying the signal may introduce a phase shift. If a variable gain is used to deal with a wide dynamic range of input signals, the variable phase shift of the variable gain stage may be considered. The resulting electrical signals may enter detection circuitry 44, which may include phase detection circuitry 45 and amplitude detection circuitry 46. The output of the detection circuitry 44 may be amplified and digitized via analog to digital converter 47 and then input into a processor (e.g., DSP 49) so that the phase and amplitude differences may be correlated to physiological information. As depicted, the system 10 includes a DSP 48 and microprocessor 49. In other embodiments, the functions of these two devices may be combined into one processor. The DSP 48 may also require separate RAM and Flash, which may be internal or external to the chip. Further, in some embodiments, the processor may include the ADC. For example, a Kinetis (Freescale Semiconductor, Inc, Austin, Tex.) processor that includes dual 16-bit SAR converters and an ARM processor may be employed in the system 10.

By analyzing changes in amplitude and phase between the received single-wavelength photon density wave signals and corresponding emitted single-wavelength photon density wave signals of a particular wavelength of light, absorption and scattering properties of the patient 26 tissue for that wavelength of light may be determined. Detection circuitry 44 may be configured to detect phase differences (via phase detection circuitry 45) and amplitude differences (via amplitude detection circuitry 46) between the reference signals and the photon density wave signals. In certain embodiments, the phase detection circuitry 45 and the amplitude detection circuitry 46 may be part of a single detection circuit. For example, the detection circuitry 44 may include one or more integrated circuit devices for measuring amplitude and phase between two independent input signals. An example of such a device is the AD8302 Gain Phase Detector (available from Analog Devices, Inc.).

To obtain phase changes corresponding to scattering in the patient 26 tissue, the detection circuitry 44 may obtain the received single-wavelength photon density wave signals from the detectors 42 and clock signals or reference signals relating to the corresponding original emitted single-wavelength photon density wave signals from the driving circuitry 28. The phase detection circuitry 45 may simultaneously detect phase changes on multiple channels of signals, or may detect phase changes by cycling through multiple channels and sampling the channels one at a time. Similarly, to obtain amplitude differences, the amplitude detection circuitry 46 may obtain the received single-wavelength photon density wave signals from the detectors 42 and clock signals or reference signals relating to the corresponding original emitted single-wavelength photon density wave signals from the driving circuitry 28. In certain embodiments, the detection circuitry 44 and the driving circuit 28 may be individual components of a single semiconductor device, such as a DVD R/W driver circuit. Such devices may include the LMH6525 (available from National Semiconductor Inc.). In other embodiments, the amplitude and/or phase detection may be handled in the digital domain (e.g., via any suitable processor, such as DSP 48).

In further examples, the received signal detected by detectors 42 are downconverted to a baseband or intermediate frequency (IF) using common communication system tuner techniques. A combined programmable gain block and downconversion block may be found in many commodity components and devices. The baseband or IF signals could then be directly digitized and transferred to the processor (e.g., DSP 48), which calculates amplitude and phase delays. A wider range of input phase relationships could be handled in this manner. A cross-correlation between the reference signal and the received PDW signals may be used to calculate phase delay via the DSP 48. Amplitude could be determined by comparing signal power. Digital filtering or conditioning could be performed on the signals prior to determination of amplitude or phase delay. In yet further examples, the processor may also determine physiological parameters from the raw signals determined by detectors 42, or the offset (DC) or time varying (AC) components of the phase and amplitude signals instead of a discrete phase detection circuitry 44. The DSP 48 may also evaluate signal quality and ambient noise and vary the drive or reference signal power, waveform shape, or frequency to increase the signal-to-noise ratio of the signals.

The output of the phase detection circuitry may be digitized via analog to digital converter (ADC) 47. Suitable ADCs 47 may include one or more sigma-delta modulators for analog-to-digital conversion that are configured to digitize a demodulated signal (e.g., via demodulator 40) with separate red and IR signals, such as the sigma-delta modulator set forth in U.S. Pat. No. 5,921,921, the specification of which is incorporated by reference herein for all purposes. Sigma delta converters generally include an anti-aliasing filter, such as a sinc³ filter, which may provide certain advantages in conversions of continuous signals but that may present certain complexities for capturing impulses or edges such as a square wave. Sigma delta converters may be appropriate for converting the phase and amplitude signals provided any multiplexing in the system (switching between different wavelengths of light or dark periods) does not create sharp discontinuities in the phase or amplitude signals that are outside the specification of the sigma delta's frequency response or that enough settling time is available after such a discontinuity for the signals to settle and be converted. Accordingly, the selection of the appropriate ADC 47 may involve considerations of signal multiplexing and data conversion. A sigma delta converter may be used to convert the baseband signals after HW demodulation or at an intermediate frequency (IF) if a device such as a radio tuner or mixer is used to downconvert the high frequency signals to a lower (intermediate) frequency, which is much easier to acquire with a data converter). Phase and amplitude can also be calculated at an intermediate frequency after mixing the received signal with a sinusoidal signal. The ADC 47 may include an audio-specific converter with amplitude digitized on the right channel and phase digitized on the left channel (or with the inputs reversed).

Other types of ADCs 47 may include flash, pipeline, and successive approximation (SAR) converters. Analog and phase may also be sampled using a voltage to frequency converter. For example, discrete counters or internal timers on the DSP or microcontroller may count the number of pulses in a specific time interval and convert the pulse count back to a voltage or amplitude/phase measurement. The ADC 47 may be discrete a data conversion device, an audio CODEC, or integrated into a semiconductor device, such as a processor, microcontroller, or the DSP 48. In one embodiment, the ADC 47 may include two sigma-delta modulators, one for the red channel and one for the IR channel. As noted, the phase and amplitude differences may be relatively low frequency compared to the reference and detected signals. Accordingly, in embodiments in which the phase difference and amplitude difference signals are digitized, the ADC 47 may include integral analog filters or digital signal processing circuitry configured to improve the signal to noise ratio for relatively low frequency signals. The ADC 47 may be configured to receive demodulated or multiplexed IR and red signals. Further, the amplitude and phase signals may be multiplexed into a single converter using a discrete analog multiplexer or a sample and hold circuit. In other embodiments, the ADC 47 may include integral multiplexing or sample and hold circuitry. In embodiments in which the ADC 47 is configured to multiplex signals (e.g., phase and amplitude or IR and red), the ADC 47 may include a digital filter with a settling time that is less than the desired multiplexing rate. In embodiments in which the phase and amplitude detection takes place in the digital domain, a very high speed converter for the received waveforms (e.g., at least 2× the highest modulation frequency) may be employed. For example, the bandwidth of the received signal is generally the modulation frequency+/−the frequency of the plethysmographic signal. For a modulation frequency Fc with a pleth bandwidth of 40 Hz, the sampling rate is at least 2×(Fc+40 Hz).

The DSP 48, or any suitable type of processor, may receive the phase change information from the phase detection circuitry 44 and reference signal information from the driver circuit 28. By comparing amplitude changes between the received single-wavelength photon density wave signals and the emitted single-wavelength photon density wave signals of the same corresponding wavelength of light, absorption properties of the patient 26 tissue for each wavelength of light may be determined. Using the absorption and scattering information associated with the amplitude changes and phase changes of the photon density wave signals passed through the patient 26, the DSP 48 may determine a variety properties based on algorithms stored in memory on the DSP 48 or received from external sources, such as a microprocessor 49 or other devices via a bus 50.

In general, the DSP 48 may ascertain certain properties of the patient 26 tissue based on the following relationships described below. For a modulation frequency where the product of the frequency and the mean time between absorption events is much larger than 1, the change in phase Δφ between two points located a distance r from each other on a tissue bed may be given by the following relation:

$$Δφ = r \sqrt{\frac{{ωμ}_{s}^{′}}{6 c}} , \qquad ( 1 )$$

where c is the speed of light in the probed medium, ω is the angular frequency of modulation, and μₛ′ is the reduced scattering coefficient. The reduced scattering coefficient for a tissue bed accounts for both blood and surrounding tissue components. This can be written as:

μₛ_{_{—}}ₜₒₜₐₗ′=V_{blood}μₛ_{_{—}}_{blood}′+Vₜᵢₛₛᵤₑμₛ_{_{—}}ₜᵢₛₛᵤₑ′ (2)

The time varying component of this equation at a single wavelength will generally be only the portion due to arterial blood. The time varying component of this equation at a second wavelength will allow for the deconvolution of the scattering coefficient. The scattering coefficient for blood is related to the hematocrit (HCT) through the following relation:

μₛ_{_{—}}_{blood}′=σₛ(1−g)(HCT/Vᵢ)(1−HCT)(1.4−HCT) (3),

where g is the anisotropy factor, σ is the scattering cross section of an erythrocyte, Vi is the volume of an erythrocyte and HCT is the hematocrit.

As indicated above, the phase of the photon density waves may be sensitive to changes in the scattering coefficient, while the amplitude of the photon density waves may be sensitive to the concentration of absorbers in the medium. Specifically, with regard to amplitude measurements, the AC amplitude and DC amplitude may yield information about absorption in the volume. Thus, detection of amplitude changes in the photon density waves may be utilized to calculate absorber concentration values in the observed medium, such as blood oxygen saturation values. Such calculations may be made using a standard ratio of ratios (e.g., ratrat) technique for the constant and modulated values of the photon density wave amplitudes at two wavelengths. Once the ratio of ratios values is obtained, it may be mapped to the saturation from clinical calibration curves. In general, the amplitude of the resulting photon density waves after passing through the patient 26 tissue may be described as follows:

$$A = \frac{{A}_{0}}{4 π {Dr}_{sd}} exp [ - {r}_{sd} \sqrt{\frac{{[ {( {μ}_{a} c )}^{2} + {ω}^{2} ]}^{\frac{1}{2}} + {μ}_{a} c}{2 D}} ] , \qquad ( 4 )$$

where A₀ is the initial amplitude, D is the diffusion coefficient given as

$$D = \frac{c}{3 ( {μ}_{s}^{′} + {μ}_{a} )} .$$

μₐ is the absorption coefficient, and r_{sd} is the distance between the emitter and the detector.

With regard to phase shift measurements, when the wavelength of the photon density waves is less than a mean absorption distance of the pulsatile tissue of the patient 26, the phase becomes almost exclusively a function of the scattering coefficient. While dependent upon the tissue bed being probed, this is generally believed to occur at a modulation frequency in the range of approximately 500 MHz. Thus, the phase shift measurement may yield information about the number of erythrocytes or red blood cells in the local probed volume. The HCT discussed above is proportional to the number of erythrocytes. Accordingly, by sweeping frequencies, a multi-parameter output may be obtained that relates to standard pulse oximetry measurements as well as the puddle hematocrit. In general, the change in phase of the resulting photon density waves after passing through the patient 26 tissue may be described as follows:

$$ΔΦ = {r}_{sd} \sqrt{\frac{{[ {( {μ}_{a} c )}^{2} + {ω}^{2} ]}^{\frac{1}{2}} - {μ}_{a} c}{D}} \qquad ( 5 )$$

The amplitude and phase at a given frequency may be proportional to the scattering and absorption coefficient at a given wavelength until the product of the frequency and the mean time between absorption events is much larger than 1. When the product of the frequency and the mean time between absorption events is much larger than 1, the amplitude is a function of the absorption and phase is only a function of the scattering. Thus, in some embodiments, the driving circuit 28 may perform a frequency sweep over time (e.g., from 100 MHz to 1 GHz) to reduce the error in the determination of a single value of reduced scattering coefficient for the blood and a single value of absorption coefficient.

In some embodiments, by modulating the light sources at a sufficient frequency, and, thus, facilitating a detectable phase shift that corresponds to scattering particles, present embodiments may provide an extra degree of certainty for blood flow parameter measurements. Indeed, the detected amplitude for the photon density waves may be utilized to calculate traditional pulse oximetry information and the phase may be utilized to confirm that such values are correct (e.g., within a certain range of error). For example, the amplitude information may be utilized to calculate a blood oxygen saturation (SpO₂) value and empirical data may indicate that a particular SpO₂ value should correspond to a particular phase variation at a given frequency. In other words, there may be a certain phase change that should accompany a given increase in absorber observed as a change in amplitude. Various known techniques (e.g., learning based algorithms such as support vector machines, cluster analysis, neural networks, and PCA) based on the measured phase shift and amplitude change may be compared to determine if the amplitude shift and phase shift correlate to a known SpO₂. If both the measured amplitude shift and phase shift correlate to a known SpO₂, the measured SpO₂ value may be deemed appropriate and displayed or utilized as a correct SpO₂ value. Alternatively, if the measured amplitude shift and phase shift do not agree, the calculated SpO₂ value may be identified as being corrupt or including too much noise and, thus, may be discarded

As shown in FIG. 2, the patient monitor 12 may include a general- or special-purpose microprocessor 49 on a bus 50, which may govern other general operations of the patient monitor 12, such as how data from the DSP 48 is employed by other components on the bus 50. A network interface card (NIC) 52 may enable the patient monitor 12 to communicate with external devices on a network. A read only memory (ROM) 54 may store certain algorithms, such as those used by the DSP 48 to determine absorption and scattering properties of the patient tissue 26, and nonvolatile storage 56 may store longer long-term data. Additionally or alternatively the nonvolatile storage 56 may also store the algorithms for determining tissue properties.

Other components of the patient monitor 12 may include random access memory (RAM) 58, a display interface 60, and control inputs 62. The RAM 58 may provide temporary storage of variables and other data employed while carry out certain techniques described herein, while the display interface 60 may allow physiological parameters obtained by the patient monitor 12 to appear on the display 20. Control inputs 62 may enable a physician or other medical practitioner to vary the operation of the patient monitor 12. By way of example, a practitioner may select whether the patient 26 is an adult or neonate, and/or whether the tissue is high perfusion or low perfusion tissue. Such a selection with the control inputs 60 may vary the modulation frequency of one or more of the single-wavelength photon density wave signals, may disable one or more of the single-wavelength photon density wave signals, or may cause a preprogrammed sequence of operation, such as a sweep of modulation frequencies for one or more of the single-wavelength photon density wave signals, to begin.

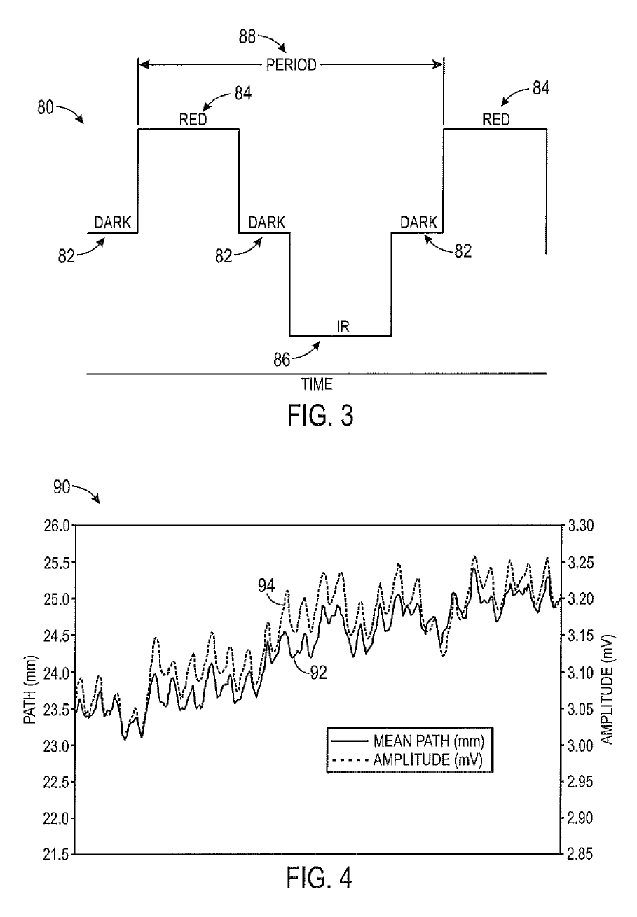

FIG. 3 illustrates an example of a source modulation signal as driven by cross-coupled light emitters (e.g., LEDs or lasers) in accordance with some embodiments. Specifically, FIG. 3 illustrates a control signal 80 that may be generated by the drive circuit 28 to activate and/or deactivate the emitter output 22, including red and IR light sources, such as a pair of laser diodes (LDs). In other embodiments, separate modulators may be utilized for each light source and/or additional light sources. Indeed, when multiple emitters are utilized, each emitter may be modulated by a separate modulator.

In the illustrated embodiment, the control signal 80 is representative of dark intervals 82, intervals of power 84 being supplied to a red LD, and intervals of power 86 being supplied to an IR LD over time. Further, the control signal 80 has a period designated by reference number 88. This period 88 may be adjusted such that each of the LDs may be modulated with a desired frequency (e.g., 50 MHz to 3.0 GHz) to generate photon density waves. Such adjustments to the modulation frequency may facilitate detection of phase shifts in the photon density waves, and, thus, variations in scattering based on such phase shifts. As may be appreciated by those of ordinary skill in the art, the control signal 80 may be adjusted or modified for different scenarios. For example, the control signal 80 may be adjusted to be generally sinusoidal, adjusted to include various intensity levels, and so forth. The sinusoidal nature of the wave may be controlled by a wave generator and the intensity levels may be adjusted by providing more power and/or by reducing dark intervals and increasing the length of time that light is emitted. Further, the characteristics of the signal 80 may be selected with regard to later conditioning steps. In addition, the signal 80 may be scaled so that the detected phase difference signal has an adequate signal-to-noise ratio. The signal may be also be scaled based on a signal quality input, e.g., from DSP. Further, the signal 80 may be used only during certain monitoring situations. For example, when the monitor 12 is in a power-saving mode, the monitor 12 may switch to conventional pulse oximetry monitoring (e.g., without a high frequency modulated light source) or the signal 80 may be scaled to conserve power. The power-saving mode may be periodic, e.g., between samples of the plethysmographic waveform. In particular embodiments, it may be assumed that the received signal comes entirely from the laser source (e.g. signals correlated with the reference) because little light modulation exists at 1 GHz. Dark periods may or may not be present in the control signal 80. Further, the control signal 80 may be of any suitable shape for multiplexing the signal in embodiments in which multiple wavelengths are used.

FIG. 4 is a graph 90 showing a mean path length 92 calculated from a phase delay signal and an amplitude difference 94 taken from a photon density wave-configured sensor. Both the mean path length 92 and amplitude difference 94 traces exhibit indications of arterial pulses and also show other sources of noise. Accordingly, both signals may be conditioned to remove noise that may obscure the signal.

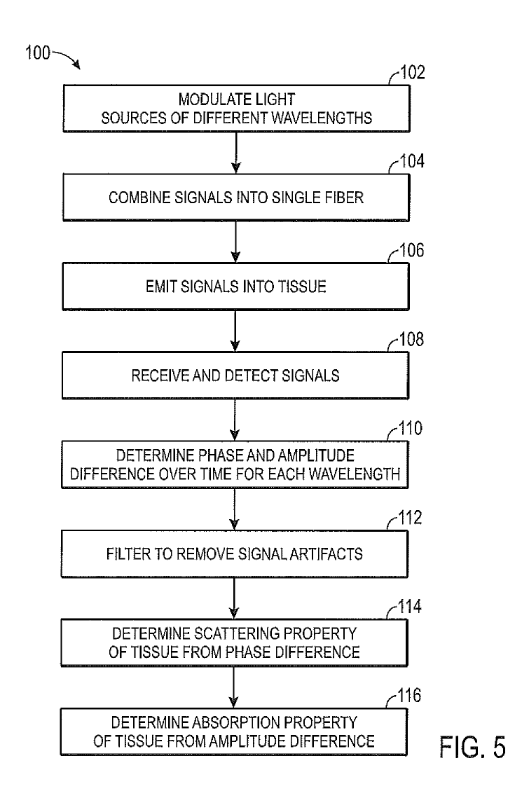

FIG. 5 is a flow diagram 100 that represents an embodiment of a method for conditioning signals representative of photon density wave measurements using two wavelengths of light. In a first step 102, the driving circuit 28 may modulate light sources of different wavelengths at modulation frequencies sufficient to produce resolvable photon density waves within the patient 26. Generally, such modulation frequencies may result in a photon density wave wavelength shorter than a mean absorption distance of the pulsatile tissue of the patient 26. In other words, such modulation frequencies may exceed the product of the mean absorption coefficient multiplied by the speed of light. Thus, depending on the patient 26, modulation frequencies may be between 50 MHz to 3 GHz. The modulation frequencies may or may not vary among the light sources and may or may not vary over time. In some embodiments, all light sources may be modulated at a frequency of approximately 500 MHz.

In step 104, the several single-wavelength photon density wave signals may be combined into a single multi-wavelength photon density wave signal via the fiber coupler 34, before being transmitted to the sensor 14 via the optical cable 36. In step 106, the multi-wavelength photon density wave signal may enter pulsatile tissue of the patient 26 through the emitter output 22 of the sensor 14. After the signal has been reflected or transmitted through the patient 26 tissue, the detector input 24 of the sensor 14 may receive and guide the signal to the optical cable 38, which may transmit the signal back to the patient monitor 12 at step 108. In step 110, phase change 4 and/or amplitude change ΔDC₁ and/or ΔAC₁ values for one of the single-wavelength components of the multi-wavelength photon density wave signal may be received into or determined by a processor, such as the DSP 48.

The digitized Δφ₁ and/or amplitude change ΔDC₁ and/or ΔAC₁ signals, for both red and IR signals, may then be conditioned to remove signal artifacts at step 112. Moreover, signals may be scaled or normalized to reduce the impact of short-term artifacts (e.g., patient motion or EMI). For example, filters or transforms including low pass, high pass, band pass, derivative, integral, cardiac-gated averaging, frequency, spectral, cepstral, wavelet transforms empirical mode decomposing and ensemble averaging may be applied to the signal. In other embodiments, rather than a fixed filter, the signal may be filtered by an adaptive filter, such as a Kalman, adaptive comb, adaptive noise canceller, joint process, root mean square, least mean squares, or lattice filter. The filter weights may be determined by one or more metrics, trends, patterns or distributions of their inputs or outputs, including signal quality metrics. In one embodiment, the red and IR signals are processed separately, but with the same filtering weights. The filtering may be delayed approximately to allow the signal metrics to be calculated first.

In an embodiment in which the signal is subjected to ensemble averaging, the filters use continuously variable weights. If samples are not to be ensemble-averaged, then the weighting for the previous filtered samples is set to zero in the weighted average, and the new samples are still processed through the code. This block tracks the age of the signal—the accumulated amount of filtering (sum of response times and delays in processing). Too old a result will be flagged (if good pulses haven't been detected for awhile).

In addition, these signals may be subjected to metrics (e.g., signal quality metrics) to determine if they are sufficiently correlated and/or reliable. For example, the digitized Δφ₁ and/or amplitude change ΔDC₁ and/or ΔAC₁ signals may be provided to a pulse identification block to identify pulses, and qualify them as likely arterial pulses. In one embodiment, this step may be performed by a pre-trained neural net, and is primarily done on the IR signal. Pulse qualities may be identified for the phase change and amplitude change traces by examining amplitude, shape and frequency. Other qualities that may be examined include pulse shape (derivative skew), period variability, pulse amplitude, average pulse period and variability, ratio of ratios variability, and frequency content relative to pulse rate. Further, the metrics may be derived from a transform (e.g., one or more filters as provided herein) or from auto or cross-correlation. The metrics may also include a comparison of the phase or amplitude differences with stored empirical or theoretical data as well as pulse or artifact models. Further signal classification techniques may include neural nets, fuzzy logic, genetic or other learning-based algorithms, which may include past data inputs. Such classification may also include inputs from other types of sensors (e.g., motion, pressure, strain, temperature, flow, impedance, or ultrasound).

Signal quality metrics may be used to determine the reliability of one or more of the phase difference or amplitude difference signals. In the case of the phase difference signal, this signal may be used when reliable. When this signal is unreliable, an estimated path length may be substituted for a path length calculated from the phase difference. In one embodiment, the signal quality or a physiological parameter may be determined based on a relationship between the phase and amplitude differences. This relationship may be determined according to desired time windows or number of pulses, and the relationship (e.g., ratio) may be assessed by linear regression, linear combination, multivariate analysis, principal component analysis, matrix analysis, or independent component analysis. Further assessment may include the application of parallel or alternate algorithms or estimation techniques, such as Hidden Markov Models, particle filters, or a combination of techniques. In other embodiments, the metrics may include inputs from other physiological parameters, monitors, or patient information. For example, the metrics may include comparing a period of phase and/or amplitude changes related to heart beats with a heart rate determined from an ECG (e.g., C-LOCK ECG synchronization) input. The metrics may also include determining a heart beat interval from an ECG input, extracting the frequency component of a phase or amplitude signal that matches the heart rate, and calculating the amplitude of the phase or amplitude signal to determine the signal strength at the heart rate.

In step 114, the DSP 48 may determine a scattering property of the patient 26 tissue for the moment in time at which the single-wavelength component of the multi-wavelength photon density wave signal has passed through the pulsatile tissue of the patient 26. Generally, the scattering property may be represented by a scattering coefficient, and may be determined based on the phase change Δφ₁ value obtained using Equation (1). In step 116, the DSP 48 may determine an absorption property of the patient 26 tissue for the moment in time at which the single-wavelength component of the multi-wavelength photon density wave signal has passed through the pulsatile tissue of the patient 26. Generally, the scattering property may be represented by an absorption coefficient, and may be determined based on the amplitude change ΔDC₁ and/or ΔAC₁ values by using Equations (1) and (4).

While the embodiments set forth in the present disclosure may be susceptible to various modifications and alternative forms, specific embodiments have been shown by way of example in the drawings and have been described in detail herein. However, it should be understood that the disclosure is not intended to be limited to the particular forms disclosed. The disclosure is to cover all modifications, equivalents, and alternatives falling within the spirit and scope of the disclosure as defined by the following appended claims.

What is claimed is:

1. A medical monitoring system, comprising:
an emitter;
a modulator configured to control the emitter with a drive signal that provides modulation frequencies in a frequency range suitable to produce photon density waves;
a detector configured to detect the photon density waves to generate a photon density wave signal;
a memory storing instructions to:
apply a fixed or adaptive filter to the photon density wave signal to generate a conditioned photon density wave signal;
determine a photon path length or phase delay based on the conditioned photon density wave signal; and
calculate a physiological parameter based at least in part on the mean photon path length; and
a processor configured to execute the instructions.

2. The system of claim 1, wherein the instructions are configured to compare the conditioned photon density wave signal to a reference signal to determine a phase shift, and wherein the mean photon path length is determined based on phase shift.

3. The system of claim 1, wherein the modulation frequency is in a range of about 50 MHz to 3 GHz.

4. The system of claim 1, wherein the filter comprises one or more of a Kalman, adaptive comb, adaptive noise cancellation, joint process, root mean square or least mean squares, or lattice filter.

5. The system of claim 1, wherein the detector is configured to determine an amplitude shift based on the conditioned photon density wave signal.

6. The system of claim 1, wherein the physiological parameter comprises a blood oxygen saturation level.

7. The system of claim 1, wherein the instructions are configured to determine a signal quality of photon density wave signal or the photon path length calculated from the photon density wave signal.

8. The system of claim 1, wherein the instructions are configured to scale or normalize an output power of the emitter based on a signal quality metric of the photon density wave signal.

9. The system of claim 1, wherein the modulator is inactive during a power-saving mode of the monitoring system.

10. The system of claim 1, wherein the emitter comprises at least a first light source configured to emit light at a first wavelength and a second light source configured to emit light at a second wavelength.

11. A method of analyzing tissue, comprising:
receiving from the tissue a signal representative of detected photon density waves;
comparing an amplitude and phase of the signal to a reference signal to determine an amplitude difference and a phase difference over time;
evaluating a reliability of the phase difference using metrics; and
determining a physiological parameter based on the phase difference and the amplitude difference when the metrics indicate the phase difference is reliable.

12. The method of claim 11, wherein determining the physiological parameter comprises determining an optical path length from the phase difference when the phase difference is reliable.

13. The method of claim 11, comprising determining the physiological parameter based on the amplitude difference and an optical path length estimate when the metrics indicate the phase difference is unreliable.

14. The method of claim 11, wherein the metrics comprise determining a pulse quality of the phase difference over time.

15. The method of claim 11, wherein the metrics comprise a pulsatile amplitude, period, or shape of the phase difference over time.

16. The method of claim 11, wherein the metrics comprise comparing the phase difference over time to stored empirical data.

17. The method of claim 11, wherein the metrics comprises comparing the amplitude difference and the phase difference over time to determine a level of correlation.

18. The method of claim 11, wherein determining the physiological parameter comprises determining a relationship between the amplitude difference and the phase difference.

19. The method of claim 11, wherein the metrics comprise comparing a period of phase changes related to heart beats with a heart rate determined from an ECG input.

20. The method of claim 11, wherein the metrics comprise comparing the period of amplitude changes related to heart beats with a heart rate determined from an ECG input.

21. The method of claim 11, wherein the metrics comprises comparing the period of amplitude and phase changes related to heart beats with a heart rate determined from an ECG input.

22. The method of claim 11, wherein the metrics comprise determining a heart beat interval from an ECG input, extracting the frequency component of a phase or amplitude signal that matches the heart rate, and calculating the amplitude of the phase or amplitude signal to determine the signal strength at the heart rate.

23. A pulse oximeter, comprising:
a modulator configured to modulate a first light source and a second light source at modulation frequencies in a frequency range suitable to produce photon density waves of at least two frequencies of light;
a detector configured to detect the photon density waves from the first light source and the second light source after the photon density waves have passed through tissue and output a multiplexed analog photon density wave signal that is indicative of a number of photons detected over a time period from the first light source and the second light source;
an analog to digital converter configured to digitize a multiplexed signal representative of the detected photon density waves from the first light source and the second light source; and
analysis circuitry configured to receive the digitized multiplexed photon density wave signal, demodulate the multiplexed signal into component parts representative of the photon density wave signal from the first light source and the second light source, and determine a physiological parameter based on a phase component and an amplitude component of the photon density wave signal from the first light source and the second light source.

24. The pulse oximeter of claim 23, wherein the modulation frequency of the first light source is different than the modulation frequency of the second light source.

25. The pulse oximeter of claim 23, wherein the multiplexed signal is time division multiplexed.
